(19) United States
(12) Patent Application Publication
(10) Pub. No.: US 2007/0224690 A1
(43) Pub. Date: Sep. 27, 2007

(54) VARENICLINE STANDARDS AND IMPURITY CONTROLS

(75) Inventors: Frank R. Busch, Gales Ferry, CT (US); Gregory J. Withbroe, Gales Ferry, CT (US); Terry G. Sinay, Shallotte, NC (US); Karen S. Bronk, Mystic, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 11/691,061

(22) Filed: Mar. 26, 2007

Related U.S. Application Data
(60) Provisional application No. 60/786,543, filed on Mar. 27, 2006.
(60) Provisional application No. 60/828,142, filed on Oct. 4, 2006.

(51) Int. Cl. G01N 33/00

(52) U.S. Cl. 436/98; 544/343

(57) ABSTRACT

The subject invention provides a varenicline composition that comprises varenicline, or a pharmaceutically acceptable salt thereof, and an amount of a compound selected from one or more of several mononitro, monoamino, mixed aminonitro, diamino or dinitro intermediates, and the concentration of said compound is greater than 0 ppm and not greater than about 500 ppm, not greater than about 100 ppm or not greater than about 10 ppm. Methods for synthesizing and using such varenicline compositions are also provided.

### BACKGROUND OF THE INVENTION

Efforts are made to prepare pharmaceutical products of a high grade and with a minimum amount of impurities present. The control of impurities requires a study of various options to decide upon the reaction conditions and testing protocols necessary to insure that drugs which are administered to the public are pure.

Guidance given by regulatory bodies, including the United States Food and Drug Administration (FDA), suggests that impurities in drugs be identified, if present, if they are at a level of 0.1% (that is, 1000 ppm) or greater for drug substances dosed at 2 g/day or lower. (Note that ppm is parts per million, so that 1%=10,000 ppm; 0.1%=1000 ppm; 0.01%=100 ppm; and 0.001%=10 ppm). For example, the FDA has indicated that identification of impurities below apparent levels of 0.1% for a 2 g/day-dosed drug substance is generally not considered necessary (Federal Register, 65(140), 45085-45090, 45086 and 45089 (Jul. 20, 2000)). However, the FDA also points out that tighter controls may be necessary for some impurities, depending upon their specific properties (Id. at 45086). Furthermore, studies to obtain safety information for a proposed quantity of an impurity are recommended if the proposed quantity exceeds a qualification threshold of 0.05% (500 ppm for a drug substance dosed at 2 g/day or lower (Id. at 45087 and 45089).

Varenicline (5,8,14-triazatetracyclo[10.3.1.0^{2.11}.0^{4.9}]-hexadeca-2(11),3,5,7,9-pentaene) having the structural formula:

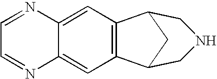

is known to bind to neuronal nicotinic acetylcholine specific receptor sites and is useful in modulating cholinergic function. This compound is useful in the treatment of inflammatory bowel disease (including but not limited to ulcerative colitis, pyoderma gangrenosum and Crohn's disease), irritable bowel syndrome, spastic dystonia, chronic pain, acute pain, celiac sprue, pouchitis, vasoconstriction, anxiety, panic disorder, depression, bipolar disorder, autism, sleep disorders, jet lag, amyotrophic lateral sclerosis (ALS), cognitive dysfunction, drug/toxin-induced cognitive impairment (e.g., from alcohol, barbiturates, vitamin deficiencies, recreational drugs, lead, arsenic, mercury), disease-induced cognitive impairment (e.g., arising from Alzheimer's disease (senile dementia), vascular dementia, Parkinson's disease, multiple sclerosis, AIDS, encephalitis, trauma, renal and hepatic encephalopathy, hypothyroidism, Pick's disease, Korsakoff's syndrome and frontal and subcortical dementia), hypertension, bulimia, anorexia, obesity, cardiac arrhythmias, gastric acid hypersecretion, ulcers, pheochromocytoma, progressive supramuscular palsy, chemical dependencies and addictions (e.g., dependencies on, or addictions to nicotine (and/or tobacco products), alcohol, benzodiazepines, barbiturates, opioids or cocaine), headache, migraine, stroke, traumatic brain injury (TBI), obsessive-compulsive disorder (OCD), psychosis, Huntington's chorea, tardive dyskinesia, hyperkinesia, dyslexia, schizophrenia, multi-infarct dementia, age-related cognitive decline, epilepsy, including petit mal absence epilepsy, attention deficit hyperactivity disorder (ADHD), Tourette's Syndrome, particularly, nicotine dependency, addiction and withdrawal; including use in smoking cessation therapy.

Compounds that bind to neuronal nicotinic receptor sites, including 5,8,14-triazatetracyclo[10.3.1.0^{2.11}.0^{4.9}]-hexadeca-2(11),3,5,7,9-pentaene, and its hydrochloride salt, are referred to in WO 99/35131, published Jul. 15, 1999 (corresponding to U.S. Ser. No. 09/402,010, filed Sep. 28, 1999 and 09/514,002, filed Feb. 25, 2000). PCT International Patent Application No. PCT/IB06/001207 discloses the derivatization of 1-(4,5-diamino-10aza-tricyclo[6.3.1.0^{2.7}]-dodecca-2-(7),3,5-trien-10-yl)-2,2,2-trifluoroethanone. PCT International Patent Application No. PCT/IB05/000351 discloses methods to control impurities in the synthetic process. The foregoing applications, owned in common with the present application and incorporated herein by reference in their entirety, generically recite pharmaceutically acceptable acid addition salts for the compounds referred to therein.

Methods of nitration and reduction of organic compounds often afford mixtures of products. It is has been discovered that such chemistry may result in mixtures of one or more of several mononitro, monoamino, mixed aminonitro, diamino or dinitro intermediates, one or more of which may comprise the unreacted starting material carried along the synthetic pathway. Undesired nitration and reduction may be controlled by varying the respective reaction conditions to afford optimized purities. In the absence of synthetic standards, the process of optimizing the process chemistry would be significantly more onerous.

Accordingly, the subject invention pertains to the techniques we have developed to control the synthesis of varenicline drug substance to insure that levels of the noted impurities are at acceptably low levels, including the preparation of synthetic standards for the optimization of the processes leading to varenicline, and pharmaceutically acceptable salts thereof.

### SUMMARY OF THE INVENTION

The present invention provides a composition comprising varenicline, or a pharmaceutically acceptable salt thereof, and an amount of a compound selected from the following:

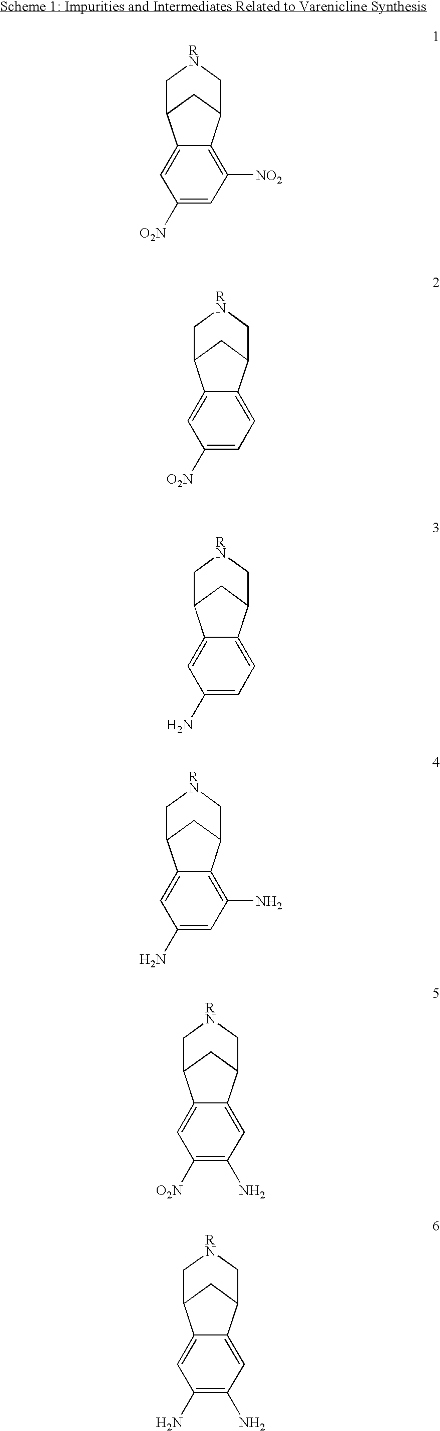

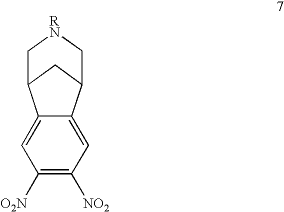

wherein R is H, acetyl or CF₃CO— and the concentration of said compound is greater than 0 ppm and not greater than about 500 ppm, not greater than about 100 ppm or not greater than about 10 ppm.

The invention also provides the composition, wherein varenicline is varenicline free base, or wherein the salt of varenicline is varenicline hydrochloride, varenicline citrate, varenicline succinate varenicline tartrate or varenicline L-tartrate. In a particular embodiment, the invention provides the composition, wherein the salt of varenicline is varenicline L-tartrate.

The invention further provides a pharmaceutical composition for treating in a mammal a disorder or condition selected from inflammatory bowel disease (including but not limited to ulcerative colitis, pyoderma gangrenosum and Crohn's disease), irritable bowel syndrome, spastic dystonia, chronic pain, acute pain, celiac sprue, pouchitis, vasoconstriction, anxiety, panic disorder, depression, bipolar disorder, autism, sleep disorders, jet lag, amyotrophic lateral sclerosis (ALS), cognitive dysfunction, drug/toxin-induced cognitive impairment (e.g., from alcohol, barbiturates, vitamin deficiencies, recreational drugs, lead, arsenic, mercury), disease-induced cognitive impairment (e.g., arising from Alzheimer's disease (senile dementia), vascular dementia, Parkinson's disease, multiple sclerosis, AIDS, encephalitis, trauma, renal and hepatic encephalopathy, hypothyroidism, Pick's disease, Korsakoff's syndrome and frontal and subcortical dementia), hypertension, bulimia, anorexia, obesity, cardiac arrhythmias, gastric acid hypersecretion, ulcers, pheochromocytoma, progressive supramuscular palsy, chemical dependencies and addictions (e.g., dependencies on, or addictions to nicotine (and/or tobacco products), alcohol, benzodiazepines, barbiturates, opioids or cocaine), headache, migraine, stroke, traumatic brain injury (TBI), obsessive-compulsive disorder (OCD), psychosis, Huntington's chorea, tardive dyskinesia, hyperkinesia, dyslexia, schizophrenia, multi-infarct dementia, age-related cognitive decline, epilepsy, including petit mal absence epilepsy, attention deficit hyperactivity disorder (ADHD), Tourette's Syndrome, comprising an amount of the composition disclosed herein effective in treating said disorder or condition and a pharmaceutically acceptable carrier. The invention also provides the pharmaceutical composition for use where the disorder or condition is nicotine dependency, addiction and withdrawal, including use in smoking cessation therapy. In a particular embodiment, the invention provides the pharmaceutical composition for use smoking cessation therapy wherein the salt of varenicline is varenicline tartrate. The invention also provides a pharmaceutical composition for smoking cessation therapy, comprising an amount of the composition set forth hereinabove effective for smoking cessation therapy and a pharmaceutically acceptable carrier. The invention also provides such a pharmaceutical composition, wherein the salt of varenicline is varenicline tartrate.

The invention also provides a method for treating in a mammal in need thereof a disorder or condition selected from inflammatory bowel disease (including but not limited to ulcerative colitis, pyoderma gangrenosum and Crohn's disease), irritable bowel syndrome, spastic dystonia, chronic pain, acute pain, celiac sprue, pouchitis, vasoconstriction, anxiety, panic disorder, depression, bipolar disorder, autism, sleep disorders, jet lag, amyotrophic lateral sclerosis (ALS), cognitive dysfunction, drug/toxin-induced cognitive impairment (e.g., from alcohol, barbiturates, vitamin deficiencies, recreational drugs, lead, arsenic, mercury), disease-induced cognitive impairment (e.g., arising from Alzheimer's disease (senile dementia), vascular dementia, Parkinson's disease, multiple sclerosis, AIDS, encephalitis, trauma, renal and hepatic encephalopathy, hypothyroidism, Pick's disease, Korsakoff's syndrome and frontal and subcortical dementia), hypertension, bulimia, anorexia, obesity, cardiac arrhythmias, gastric acid hypersecretion, ulcers, pheochromocytoma, progressive supramuscular palsy, chemical dependencies and addictions (e.g., dependencies on, or addictions to nicotine (and/or tobacco products), alcohol, benzodiazepines, barbiturates, opioids or cocaine), headache, migraine, stroke, traumatic brain injury (TBI), obsessive-compulsive disorder (OCD), psychosis, Huntington's chorea, tardive dyskinesia, hyperkinesia, dyslexia, schizophrenia, multi-infarct dementia, age-related cognitive decline, epilepsy, including petit mal absence epilepsy, attention deficit hyperactivity disorder (ADHD), Tourette's Syndrome, particularly, nicotine dependency, addiction and withdrawal; including use in smoking cessation therapy, which method comprises administering to said mammal an amount of a composition disclosed herein effective in treating said disorder or condition. In a particular embodiment, the invention provides the method for treating nicotine dependency, addiction and withdrawal, including smoking cessation therapy. In one embodiment, the method is carried out, wherein the salt of varenicline is varenicline tartrate.

The invention further provides a compound selected from the following:

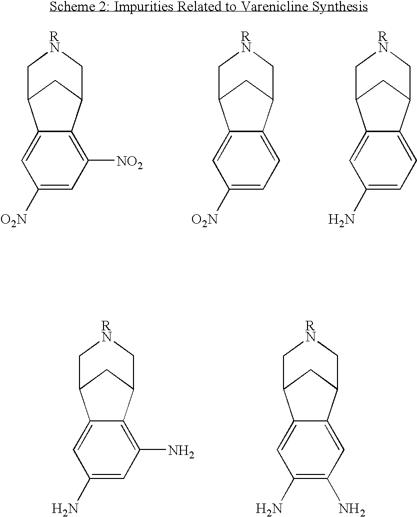

wherein R is H (designated a), acetyl (designated b) or CF₃CO-(designated c), all of which are useful as standards for the controlled synthesis of varenicline.

The level of each impurity in a sample of a batch of varenicline can be determined using standard analytical techniques known to those of ordinary skill in the art. For example, the level of one or more of the several mononitro, monoamino, mixed aminonitro, diamino or dinitro intermediates impurities noted above may be determined by normal phase HPLC, reverse phase HPLC, or gas chromatography methods.

The terms “treatment”, “treating”, and the like, refer to reversing, alleviating, or inhibiting the progress of the disorder or condition to which such term applies, or one or more symptoms of such disorder or condition. As used herein, these terms also encompass, depending on the condition of the patient, preventing the onset of a disorder or condition, or of symptoms associated with a disorder or condition, including reducing the severity of a disorder or condition or symptoms associated therewith prior to affliction with said disorder or condition. Thus, “treatment”, as used herein, can refer to administration of a compound of the invention to a subject that is not at the time of administration afflicted with the disorder or condition. “Treating” thus also encompasses preventing the recurrence of a disorder or condition or of symptoms associated therewith.

“Mammal”, as used herein, and unless otherwise indicated, means any mammal. The term “mammal” includes, for example and without limitation, dogs, cats, and humans.

The term “about”, when used herein in, for example, “less than ‘about’ 500 ppm” means within a range of plus or minus 10% of the value to which the term is being applied.

### DETAILED DESCRIPTION OF THE INVENTION

One method for synthesizing varenicline, as noted above, is taught in U.S. Pat. No. 6,410,550, the contents of which have been hereby incorporated herein by reference. One control strategy identified by the present inventors was determining the purging of the above-noted impurities during the chemical synthesis of varenicline, and then setting sufficient limits on the quality of the starting reactant used to synthesize varenicline. During a synthesis of varenicline, one or more of the intermediates undergoes extraction and crystallization during the reaction work-ups. Although each intermediate and its corresponding impurity are structurally similar and have similar solubility, there are slight differences in the physio-chemical properties. Thus, purging experiments may be conducted to determine the amount of impurity that is removed by the extraction, crystallization, and recrystallization and other processing operations.

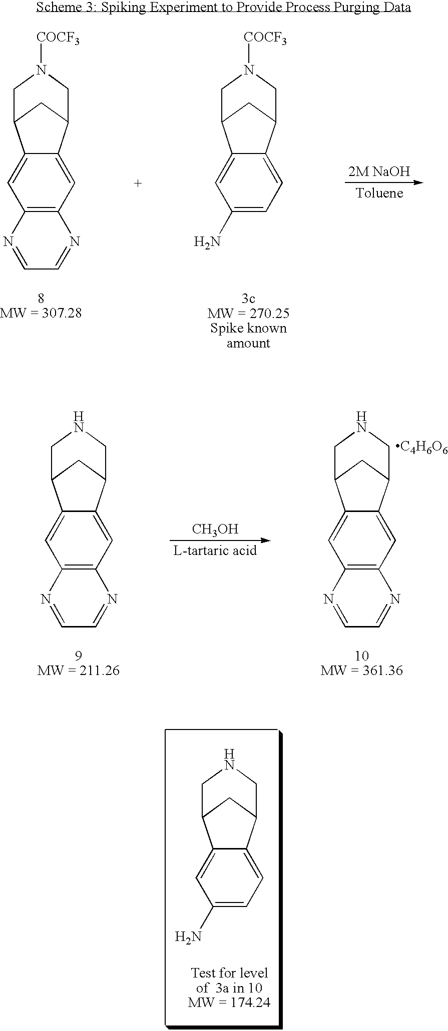

For example, referring to Scheme 3, compound 3 (R═COCF₃), a potential impurity in a batch of the starting material compound 8, may proceed under reaction conditions (2M NaOH/toluene) to result in the corresponding impurity compound 3 (R═H) in the varenicline produced. A greater than two-fold purge of compound 3a (R═COCF₃), and its subsequent analogs was found during the reaction and isolation conditions of the synthetic process. This same spiking experiment design was similarly conducted for each of the other impurities shown in Scheme 3. As noted, the impurities set forth above can be detected by standard analytical techniques when present at greater than 500 ppm. This invention pertains to the detection of these impurities at levels not greater than 500, 100 or 10 ppm. Methods to determine low levels (not greater than about 500, 100 or 10 ppm) are described below.

Analytical methodology for detecting impurities at levels not greater than about 500 ppm, or not greater than about 100 ppm, or not greater than about 10 ppm, in the protected intermediate is outlined below:

| Instrumentation: | HPLC with single quad mass spectrometer detector | | |
|---|---|---|---|
| HPLC Column: | Phenomenex Polar RP 2.0 mm × 150 mm; 4 μm particle size | | |
| Column Temperature: | 40° C. | | |
| Injection Volume: | 25 μL | | |
| Mobile Phase: | A: 0.2% Formic Acid, pH 3 with ammonium hydroxide | | |
| | B: Acetonitrile | | |
| | Time | % A | % B |
| Gradient: | 0 | 80 | 20 |
| | 5 | 80 | 20 |
| | 34 | 50 | 50 |
| | 35 | 50 | 50 |
| | 35.1 | 80 | 20 |

| | |
|---|---|
| Detection: | MSD, Selective Ion Monitoring (Optimum SIM Ions for each compound are |
| | variable; must be determined for each mass spectrometer) |
| Sample Preparation: | The protected intermediate is prepared at 2 mg/mL in 50/50 |
| | Acetonitrle/Water |
| Standard Preparation: | Impurities are prepared at 0.0002 mg/mL (100 ppm relative to |
| | sample concentration) and 0.00002 mg/mL (10 ppm relative to sample concentration) |
| Method Range: | 10 ppm to 1000 ppm |
| Method LOQ: | 10 ppm |
| Method LOD: | 1 ppm |

Analytical methodology for detecting impurities at levels not greater than about 500 ppm, or not greater than about 100 ppm or not greater than about 10 ppm, in the active pharmaceutical ingredient is outlined below:

System 1 (used for detection of compounds 3 (R = H), 4 (R = H), and 6 (R = H))

| Instrumentation: | HPLC with single quad mass spectrometer detector | | |
|---|---|---|---|
| HPLC Column: | Waters Atlantis dC₁₈ 4.6 mm × 250 mm; 5 μm particle size | | |
| Column Temperature: | 30° C. | | |
| Flow Rate: | 1.0 mL/min | | |
| Injection Volume: | 100 μL | | |
| Mobile Phase: | A: 20 mM ammonium acetate:acetonitrile (95:5, v/v) | | |
| | B: 20 mM ammonium acetate:acetonitrile (50:50, v/v) | | |
| | Time | % A | % B |
| Gradient: | 0 | 100 | 0 |
| | 22 | 100 | 0 |
| | 22.01 | 0 | 100 |
| | 27 | 0 | 100 |
| | 27.01 | 100 | 0 |
| | 34 | 100 | 0 |

| | |
|---|---|
| Detection: | MSD, Selective Ion Monitoring (Optimum SIM Ions for each compound are |
| | variable, and must be determined for each mass spectrometer) |
| Sample Preparation: | The API is prepared in 95/5 100 mM Ammonium |
| | Acetate/Acetonitrile at 15 mgA/mL |
| Standard Preparation: | Impurities are prepared in 95/5 100 mM Ammonium |
| | Acetate/Acetonitrile at 0.00015 mg/mL (10 ppm relative to sample concentration) with |
| | 15 mgA/mL of the API working standard |
| Method Range: | 10 ppm to 100 ppm |
| Method LOQ: | 10 ppm |
| Method LOD: | 1 ppm |

System 2 (used for detection of compounds 1 (R = H), 2 (R = H), 5 (R = H), and 7 (R = H))

| | |
|---|---|
| Instrumentation: | HPLC with single quad mass spectrometer detector |
| HPLC Column: | Waters Atlantis dC₁₈ 4.6 mm × 250 mm; 5 μm particle size |
| Column Temperature: | 30° C. |
| Flow Rate: | 1.0 mL/min |
| Injection Volume: | 100 μL |
| Mobile Phase: | 20 mM ammonium acetate:acetonitrile (76:24, v/v) |
| Run Time: | 22 minutes |
| Detection: | MSD, Selective Ion Monitoring (Optimum SIM Ions for each compound are |
| | variable; must be determined for each mass spectrometer) |
| Sample Preparation: | The API is prepared in System 2 mobile phase at 15 mgA/mL |
| Standard Preparation: | Impurities are prepared in System 2 mobile phase at 0.00015 mg/mL |
| | (10 ppm relative to sample concentration) with 15 mgA/mL of the API working |
| | standard |
| Method Range: | 10 ppm to 100 ppm |
| Method LOQ: | 10 ppm |
| Method LOD: | 1 ppm |

The varenicline drug substance of this invention may be administered as a pharmaceutical drug as indicated herein as described in, for example, U.S. Pat. No. 6,410,550, supra. Administration to a mammalian subject, including a human, may be alone or, preferably, in combination with pharmaceutically acceptable carriers or diluents in a pharmaceutical composition, in accordance with standard pharmaceutical practice. The pharmaceutical compositions may be administered orally or parenterally including intravenously or intramuscularly. Suitable pharmaceutical carriers include solid diluents or fillers, and sterile aqueous solutions and various organic solvents. The pharmaceutical compositions are then readily administered in a variety of dosage forms, such as tablets, powders, lozenges, syrups, and injectable solutions. These pharmaceutical compositions, if desired, may contain additional ingredients such as flavorings, binders and excipients. Thus, for purposes of oral administration, tablets containing various excipients such as sodium citrate, calcium carbonate and calcium phosphate may be employed along with various disintegrants such as starch, alginic acid and certain complex silicates, together with binding agents such as polyvinylpyrrolidone, sucrose, gelatin and acacia. Additionally, lubricating agents such as magnesium stearate, sodium lauryl sulfate and talc are often useful for tabletting purposes. Solid materials of a similar type may also be employed as fillers in soft and hard filled gelatin capsules. Preferred materials for this include lactose or milk sugar and high molecular weight polyethylene glycols. When aqueous suspensions or elixirs are desired for oral administration, the varenicline drug substance therein may be combined with various sweetening or flavoring agents, coloring matter or dyes and, if desired, emulsifying or suspending agents, together with diluents such as water, ethanol, propylene glycol, glycerin and combinations thereof.

For parenteral administration, solution or suspension of the varenicline drug substance in sesame or peanut oil, aqueous propylene glycol, or in sterile aqueous solution may be employed. Such aqueous solutions should be suitably buffered if necessary and the liquid diluent first rendered isotonic with sufficient saline or glucose. These particular aqueous solutions are especially suitable for intravenous, intramuscular, subcutaneous and intraperitoneal administration. The sterile aqueous media employed are all readily available by standard techniques known to those skilled in the art.

The effective dosage of varenicline depends on the intended route of administration and other factors such as the indication being treated and the age and weight of the subject, as generally known. In general, a daily dosage will be in the range of from about 0.25 mg of varenicline drug substance to about 200 mg, in single or divided doses, preferably from about 0.5 mg to about 20 mg per day. A typical daily dose based on a weight of about 70 kg for a patient is preferably from about 0.5 mg twice per day to about 2 mg varenicline drug substance twice per day, more preferably about 0.5 mg twice per day to about 1 mg twice per day. However, it is appreciated that the dose and dosing regimen of varenicline drug substance may be varied from the aforementioned ranges and regimens by a physician of ordinary skill in the art, depending on the particular circumstances of any specific patient.

The following Example illustrate the present invention. It is to be understood, however, that the invention, as fully described herein and as recited in the claims, is not intended to be limited by the details of the following Example.

### EXAMPLES

### Part A: Example Purge Experiment

### Experimental Determination of Purge Factor: Synthesis of Varenicline

Compound 8 was slurried in toluene and added to a solution of NaOH (3.1 equiv) in water. The biphasic mixture was warmed to 37-40° C., which produced two pale yellow clear layers. At this time 50 ppm of compound 3 (R═H) was spiked into the reaction mixture. The conversion of compound 8 to varenicline (9) was then carried out, including Darco KB-B treatment of the toluene/aqueous biphasic, and the compound 9/toluene solution was recovered. The compound 9/toluene solution was azeotropically distilled with MeOH. The resulting the compound 9/methanol solution was added to a L-(+)-tartaric acid/MeOH solution to form salt 10. After granulation, salt 10 was filtered and dried.

The isolated material was analyzed for residual compound 3 (R═H). The 50 ppm spike of 3 was introduced into the reaction mix when the temperature of 37-40° C. was reached. The compound 8 lot that was in solution already contained 6 ppm of 3 thus the total amount of 3 was 56 ppm. During deprotection, compound 3 (R═COCF₃) contained in the ingoing compound 8 was converted to the corresponding compound 3 (R═H). The final salt 10 produced was analyzed for the presence of compound 3 (R═H). The finding of 12 ppm of compound 3 (R═H) detected corresponds to a 4.7× purge factor.

### Part B: Synthesis of Standards

### 2. Synthesis of: 2,3,4,5-tetrahydro-1H-1,5-methano-3-benzazepin-7-amine

### 2a. Synthesis of 7-nitro-3-(trifluoroacetyl)-2,3,4,5-tetrahydro-1H-1,5-methano-3-benzazepine

Trifluoromethane sulfonic acid (19.8 g) was dissolved in CH₂Cl₂ (275 ml) and cooled to 0° C. Fuming HNO₃ (2.8 ml) was added dropwise. The solution was cloudy for a brief period, then went to a clear yellow solution again. In a separate vessel, 3-(trifluoroacetyl)-2,3,4,5-tetrahydro-1H-1,5-methano-3-benzazepine (15.3 g) was dissolved in CH₂Cl₂ (350 ml) and transferred to an addition funnel. This solution was added dropwise over 1 hour. The reaction temperature was maintained at 0-5° C. throughout the addition. Once addition was complete, the pot was kept at 0-5° C. for 2 hours, slowly warmed to room temperature, then stirred overnight. After TLC (eluent=1:1 EtOAc:Hex) confirmed that the reaction was complete, the reaction mixture was slowly added to ice water to quench the reaction, and stirred 20 minutes. The biphasic system was transferred to a separatory funnel for phase separation. The CH₂Cl₂/product solution was collected, simultaneously treated with Na₂SO₄ and Darco KB-B, filtered through a pad of celite and rinsed with CH₂Cl₂. The filtrate was collected and vacuum stripped to a dark yellow oil, which solidified to a waxy yellow solid on standing. The solid was allowed to dry in a vacuum oven at 45° C. overnight. This afforded crude product as a yellow solid (17.9 g of 90% pure as determined by HPLC).

The crude product (17.9 g) was taken up in EtOH (90 ml) and brought to reflux, which resulted in a clear yellow solution. The solution was allowed to slowly cool to room temperature and stir overnight. This gave a cream-colored slurry. The slurry was filtered onto a #2 Whatman paper filter and rinsed with EtOH. The solid was then allowed to dry overnight in a vacuum oven at 45° C. This afforded 14.6 g (81.1% yield) of a cream-colored solid.

### b. Synthesis of 3-(trifluoroacetyl)-2,3,4,5-tetrahydro-1H-1,5-methano3-benzazepin-7-amine

In a 500 ml glass Parr bottle, 7-nitro-3-(trifluoroacetyl)-2,3,4,5-tetrahydro-1H-1,5-methano-3-benzazepine (10 g) was slurried in MeOH (200 ml). The 50% wet 5% palladium on carbon (0.3 g) was added and the system put on the Parr shaker hydrogenation apparatus. The system was purged with N₂ (3×20 psi). The system was then purged with H₂ (3×20 psi). The system was pressurized to 45-50 psi H₂ and allowed to hydrogenate for 3 hrs. The system was depressurized and checked for reaction completion. After thin layer chromatography (eluent=1:1 EtOAc:Hex) confirmed reaction completion, the product mixture was filtered through a pad of celite followed by filtration through a 0.45 m Millipore filter to remove residual carbon. The filtrate was collected and vacuum stripped to a orange oil. The oil was allowed to dry in a vacuum oven at 45° C. overnight. This afforded 8.9 g (98.9% yield) of an orange oil.

### c. Synthesis of 2,3,4,5-tetrahydro-1H-1,5-methano-3-benzazepin-7-amine

The product from the reaction above (8.9 g) was dissolved in CH₃CN (90 ml). A solution of NaOH (7.9 g) in H₂O (25 ml) was added and the solution warmed to 70° C. for 4 hours. After thin layer chromatography (eluent=7:3 EtOAc:Hex) confirmed reaction completion, the product mixture was vacuum stripped to a light brown solid. CH₂Cl₂ (200 ml) was added and the mixture vigorously stirred for 1 hour to extract the desired product. The solids (NaOH) were filtered off and the filtrate collected. The filtrate was dried with Na₂SO₄, filtered and vacuum stripped again to a orange-brown oil. CH₂Cl₂ (25 ml) was added and the mixture allowed to stir 1 hour, which produced a tan slurry. The slurry was filtered onto a #2 Whatman filter paper and rinsed with a small amount of CH₂Cl₂. The tan solid was allowed to dry in a vacuum at 45° C. overnight. This afforded 3.0 g (52.3% yield) of a tan solid.

### 3. Synthesis of 7,8-dinitro-2,3,4,5-tetrahydro-1H-1,5-methano-3-benzazepine

7,8-Dinitro-3-(trifluoroacetyl)-2,3,4,5-tetrahydro-1H-1,5-methano-3-benzazepine (15 g), obtained from the commercial supply of this compound, was dissolved in CH₃CN (150 ml). A solution of NaOH (10.4 g) in H₂O (45 ml) was added. The mixture was warmed to 70° C. for 4 hours, slowly cooled to room temperature, then allowed to stir overnight. After thin layer chromatography (eluent=7:3 EtOAc:Hex) confirmed reaction completion, the product mixture was vacuum distilled until an aqueous slurry was achieved (pH=12.5 at this point). 2M HCl (aq) was added to adjust the pH to 7.5. The slurry was then allowed to granulate for 1 hour, filtered onto a #2 Whatman paper filter, and rinsed with H₂O. The rust-colored solid was allowed to dry under a stream of N₂ for 1 hour followed by drying overnight in a vacuum oven at 45° C. This afforded 9.1 g (84.2% yield) of a rust-colored solid.

### 4. Synthesis of 8-nitro-2,3,4,5-tetrahydro-1H-1,5-methano-3-benzazepin-7-amine

### 4a. Synthesis of: 3-(trifluoroacetyl)-8-nitro-2,3,4,5-tetrahydro-1H-1,5-methano-3-benzazepin-7-amine

According to the procedure shown in Example 2B, above, 7,8-dinitro-3-(trifluoroacetyl)-2,3,4,5-tetrahydro-1H-1,5-methano-3-benzazepine was reduced, except following the purge the system was pressurized to 25 psi H₂ and allowed to hydrogenate for 2 hrs. The system was depressurized and the slurry filtered onto a #2 Whatman filter paper. The solids were collected. This procedure was repeated three times and the crude products collected and combined (5.6 g in total). This was dissolved in CH₃CN, and filtered through a pad of celite to remove the spent catalyst. The filtrate was collected and vacuum stripped to give a yellow-brown solid. Analysis showed the desired 3-(trifluoroacetyl)-8-nitro-2,3,4,5-tetrahydro-1H-1,5-methano-3-benzazepin-7-amine to contain ˜3% of compound 6c. Yield: 5.6 g.

### 4b. Synthesis of: 8-nitro-2,3,4,5-tetrahydro-1H-1,5-methano-3-benzazepin-7-amine

3-(Trifluoroacetyl)-8-nitro-2,3,4,5-tetrahydro-1H-1,5-methano-3-benzazepin-7-amine (5.6 g) was slurried in CH₃CN (56 ml). A solution of NaOH (4.3 g) in H₂O (40 ml) was added. The mixture was warmed to 70° C. and allowed to stir for 3 hours (the mixture remained a slurry the entire duration of reaction). After thin layer chromatography (eluent=7:3 EtOAc:Hex) confirmed reaction completion, the product mixture was allowed to cool and granulate for 1 hour. The yellow-orange solids were filtered onto a #2 Whatman filter paper and rinsed with H₂O. The solid was dried under a stream of N₂ overnight. This afforded 3.6 g (92.3% yield) of a yellow-orange solid.

### 5. Synthesis of 2,3,4,5-tetrahydro-1H-1,5-methano-3-benzazepine-6,8-diamine

### 5a. Isolation of 3-(trifluoroacetyl)-6,8-Dinitro-2,3,4,5-tetrahydro-1H-1,5-methano-3-benzazepine

The meta-dinitro regio-isomer is formed as an impurity in the reaction to form 7,8-dinitro-3-(trifluoroacetyl)-2,3,4,5-tetrahydro-1H-1,5-methano-3-benzazepine, per the synthesis described in U.S. Pat. No. 6,410,550. Mother liquor waste from the reaction were purified via fractional crystallization to provide a sample of 3-(trifluoroacetyl)-6,8-Dinitro-2,3,4,5-tetrahydro-1H-1,5-methano-3-benzazepine, 53.8 g as a cream colored solid.

### 5b. Synthesis of 6,8-Dinitro-2,3,4,5-tetrahydro-1H-1,5-methano-3-benzazepine

In a manner similar to that shown in Example 2c, above, the trifluoroacetyl group was removed via base hydrolysis, followed by filtration to provide a filtrate which was collected and vacuum stripped to a damp yellow solid. H₂O (50 ml) was added and the mixture allowed to granulate for 1 hour. The yellow solid was filtered onto a #2 Whatman filter paper, rinsed with H₂O, and allowed to dry in a vacuum oven at 45° C. overnight. This afforded 7.5 g (90.4% yield) of a yellow solid.

### 5c. Synthesis of 2,3,4,5-tetrahydro-1H-1,5-methano-3-benzazepine-6,8-diamine hyrdochloride

By reduction as described in Example 2b, above, 8.0 g of 6,8-dinitro-2,3,4,5-tetrahydro-1H-1,5-methano-3-benzazepine was reduced. Following filtration the filtrate was collected, simultaneously treated with Na₂SO₄ and Darco KB-B, and refiltered though a pad of celite. The filtrate was vacuum distilled down to a sticky orange semi-solid (6.1 g theoretical), as the solid was sticky, it was reisolated from MeOH (5 ml) and CH₂Cl₂ (35 ml) by addition of HCl(g) prepared by bubbling into ice cold isopropyl ether (35 ml) to generate the hydrochloride salt, a light brown solid.

Each of the standards prepared was evaluated by standard HPLC and the respective LC/MS methods described in the text and found to be suitable for use as standards.

What is claimed is:

1. A composition comprising varenicline, a protected form of varenicline, or a pharmaceutically acceptable salt thereof, and an amount of a compound selected from the following: 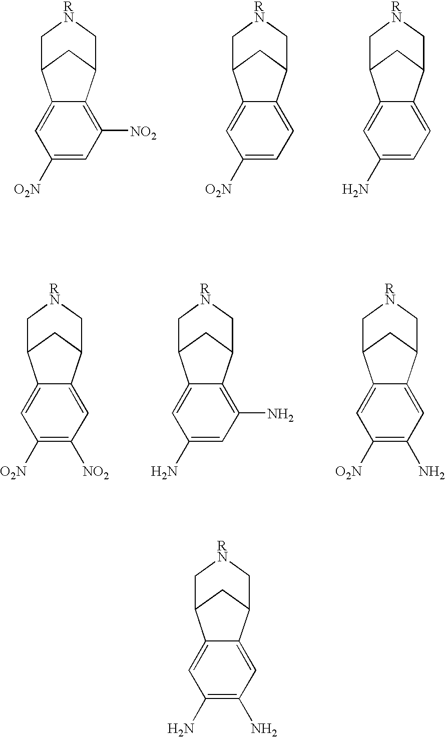
wherein R is H, acetyl or CF₃CO—, and the concentration of said compound is greater than 0 ppm and not greater than about 500 ppm, not greater than about 100 ppm or not greater than about 10 ppm.

2. The composition according to claim 1, wherein varenicline is varenicline free base, or wherein the salt of varenicline is varenicline hydrochloride, varenicline citrate, varenicline succinate or varenicline tartrate.

3. The composition according to claim 1, wherein the salt of varenicline is varenicline tartrate.

4. A pharmaceutical composition for treating a mammal suffering from a disorder or condition selected from inflammatory bowel disease, ulcerative colitis, pyoderma gangrenosum, Crohn's disease, irritable bowel syndrome, spastic dystonia, chronic pain, acute pain, celiac sprue, pouchitis, vasoconstriction, anxiety, panic disorder, depression, bipolar disorder, autism, sleep disorders, jet lag, amyotrophic lateral sclerosis (ALS), cognitive dysfunction, drug/toxin-induced cognitive impairment, disease-induced cognitive impairment, hypertension, bulimia, anorexia, obesity, cardiac arrhythmias, gastric acid hypersecretion, ulcers, pheochromocytoma, progressive supramuscular palsy, chemical dependencies and addictions, nicotine dependency, addiction and withdrawal; dependencies on, or addictions to alcohol, benzodiazepines, barbiturates, opioids or cocaine, headache, migraine, stroke, traumatic brain injury (TBI), obsessive-compulsive disorder (OCD), psychosis, Huntington's chorea, tardive dyskinesia, hyperkinesia, dyslexia, schizophrenia, multi-infarct dementia, age-related cognitive decline, epilepsy, including petit mal absence epilepsy, attention deficit hyperactivity disorder (ADHD), and Tourette's Syndrome, said composition comprising an amount of the composition of claim 1 effective in treating said disorder or condition and a pharmaceutically acceptable carrier.

5. The pharmaceutical composition according to claim 4, wherein the disorder or condition is nicotine dependency, addiction or withdrawal.

6. The pharmaceutical composition according to claim 5, wherein the salt of varenicline is varenicline tartrate.

7. A pharmaceutical composition for smoking cessation therapy, comprising an amount of the composition of claim 1 effective for smoking cessation therapy and a pharmaceutically acceptable carrier.

8. The pharmaceutical composition according to claim 7, wherein the salt of varenicline is varenicline tartrate.

9. A method for treating a mammal suffering from a disorder or condition a disorder or condition selected from inflammatory bowel disease, ulcerative colitis, pyoderma gangrenosum, Crohn's disease, irritable bowel syndrome, spastic dystonia, chronic pain, acute pain, celiac sprue, pouchitis, vasoconstriction, anxiety, panic disorder, depression, bipolar disorder, autism, sleep disorders, jet lag, amyotrophic lateral sclerosis (ALS), cognitive dysfunction, drug/toxin-induced cognitive impairment, disease-induced cognitive impairment, hypertension, bulimia, anorexia, obesity, cardiac arrhythmias, gastric acid hypersecretion, ulcers, pheochromocytoma, progressive supramuscular palsy, chemical dependencies and addictions, nicotine dependency, addiction and withdrawal; dependencies on, or addictions to alcohol, benzodiazepines, barbiturates, opioids or cocaine, headache, migraine, stroke, traumatic brain injury (TBI), obsessive-compulsive disorder (OCD), psychosis, Huntington's chorea, tardive dyskinesia, hyperkinesia, dyslexia, schizophrenia, multi-infarct dementia, age-related cognitive decline, epilepsy, including petit mal absence epilepsy, attention deficit hyperactivity disorder (ADHD), and Tourette's Syndrome, said method comprising administering to said mammal an amount of a composition of claim 1 effective in treating said disorder or condition.

10. The method according to claim 9, wherein the disorder or condition is nicotine dependency, addiction and withdrawal.

11. The method according to claim 9, wherein the salt of varenicline is varenicline tartrate.

12. A method for smoking cessation therapy, comprising administering an amount of the composition of claim 1 effective for smoking cessation therapy and a pharmaceutically acceptable carrier.

13. The method according to claim 12, wherein the salt of varenicline is varenicline tartrate.

14. A compound selected from the following: 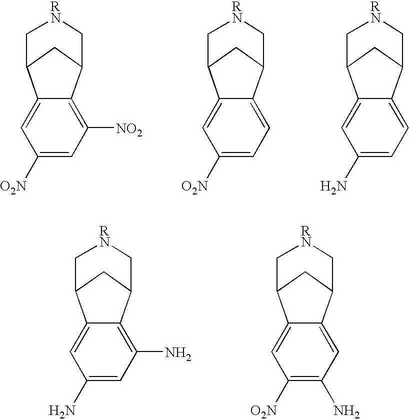
wherein R is H, acetyl or CF₃CO—.

15. The compound of claim 14, wherein R is H and an amino group is present, or an acid addition salt thereof, wherein said salt is selected from the group consisting of hydrochloride and tartrate salts.

16. The compound of claim 14, where the compound is used as an analytical standard in the manufacture of varenicline tartrate.

17. The compound of claim 14, wherein R is CF₃CO— the compound is used as an analytical standard in the manufacture of varenicline tartrate.
